(19) United States
(12) Patent Application Publication
(10) Pub. No.: US 2022/0151993 A1
(43) Pub. Date: May 19, 2022

(54) TREATMENT OF ANXIETY AND DEPRESSION

(71) Applicant: NEW YORK UNIVERSITY, New York, NY (US)

(72) Inventors: Stephen Ross, New York, NY (US); Gabrielle Agin-Liebes, San Francisco, CA (US)

(21) Appl. No.: 17/527,361

(22) Filed: Nov. 16, 2021

Related U.S. Application Data
(60) Provisional application No. 63/114,022, filed on Nov. 16, 2020.

(51) Int. Cl. A61K 31/4045; A61K 31/675; A61P 25/24; A61P 25/22

(52) U.S. Cl. CPC A61K 31/4045; A61P 25/22; A61P 25/24; A61K 31/675

(57) ABSTRACT

The invention relates to a method of alleviating depression and/or anxiety in a cancer patient, the method comprising administering a single dose of psilocin or a prodrug of psilocin, or a pharmaceutically acceptable salt thereof, to said cancer patient optionally in conjunction with a psychotherapy treatment. The method is useful in alleviating the depression and/or anxiety for at least one year. The method is also useful in reducing existential distress symptoms such as death anxiety, hopelessness and demoralization. Kits useful in the method of the invention are also described.

### FIELD OF THE INVENTION

The present invention relates to a method of alleviating depression and/or anxiety in a cancer patient using a psychedelic compound.

### BACKGROUND OF THE INVENTION

Cancer is a leading cause of morbidity and mortality globally, with approximately 14 million new diagnoses made annually. Despite technological advancements that have led to earlier detection and significantly improved medical treatments for cancer, the diagnosis still provokes intense fear and distress among many patients. It is therefore common for cancer patients to develop psychiatric distress and rates of anxiety and depressive disorders in cancer patients are high.

A common approach to the treatment of anxiety and depression is the use of antidepressants. However, the evidence of the effect of antidepressants in treating anxiety and depression in cancer patients is limited and inconsistent. Several placebo-controlled trials of antidepressants have failed to demonstrate a clear effect over placebo in cancer patients (Laoutidis et al. (2013), “Antidepressants in the treatment of depression/depressive symptoms in cancer patients: A systematic review and meta-analysis”, BMC Psychiatry, 13: 140).

The compound psilocybin has been investigated as a potential treatment for psychiatric distress (anxiety, depression) in cancer patients. Psilocybin (4-phosphoryloxy-N,N-dimethyltryptamine) is a tryptamine serotoninergic psychedelic. The IUPAC name of psilocybin is [3-(2-dimethylaminoethyl)-1H-indol-4-yl] dihydrogen phosphate. The structure of psilocybin is shown below.

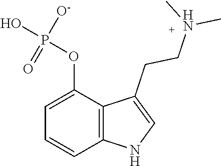

Psilocybin is metabolised in the body to psilocin (4-hydroxy-N,N-dimethyltryptamine), which exerts its effects primarily via 5HT_{2A} agonism. The structure of psilocin is below.

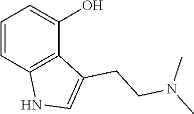

Grob et al. (2011), “Pilot study of psilocybin treatment for anxiety in patients with advanced-stage cancer”, Arch Gen Psychiatry, 68: 71-78 reports the results of a small scale study investigating psilocybin treatment for anxiety in patients with advanced-stage cancer. This study showed that there were no clinically significant adverse events associated with the administration of a single moderate dose of psilocybin (0.2 mg/kg), and that levels of anxiety were reduced up to 3 months after treatment while levels of depression were reduced up to 6 months after treatment.

Griffiths et al. (2016), “Psilocybin produces substantial and sustained decreases in depression and anxiety in patients with life-threatening cancer: A randomized double-blind trial”, J Psychopharmacol, 30: 1181-1197 reports the results of another trial. In that trial, about 80% of participants continued to show decreases in depressed mood and anxiety at the 6-month follow up.

Ross S et al. (2016), “Rapid and sustained symptom reduction following psilocybin treatment for anxiety and depression in patients with life-threatening cancer: A randomized controlled trial”, J Psychopharmacol, 30: 1165-1180 describes the results of a crossover study designed to investigate the efficacy of a single psilocybin dosing session (0.3 mg/kg), administered in conjunction with psychotherapy, to treat clinically significant anxiety or depression in patients with life-threatening cancer. In that study, rapid and sustained anxiolytic and antidepressant effects were seen up to 7 weeks post dosing. At the final 6.5 month follow up point, 60-80% of patients met the criteria for clinically significant antidepressant or anxiolytic responses.

However, despite the promising evidence regarding the acute therapeutic effects of psilocybin, there is little data suggesting safety and efficacy of these interventions in the long-term. Cancer is a chronic condition and cancer treatments can last several years. There is accordingly a need to develop interventions for the treatment of psychiatric and existential distress in cancer patients that have long-lasting effects and which can persist for the duration of the cancer diagnosis and/or cancer treatment.

### SUMMARY OF THE INVENTION

It is a finding of the present invention that a single dose of a prodrug of psilocin (for instance psilocybin) is effective in alleviating depression and/or anxiety in a cancer patient for at least one year. This finding is of great clinical and psychological significance: treatment with a psilocin prodrug can achieve sustained relief from cancer-related psychiatric distress. Advantageously, only a single dose is required which means that it is not necessary for the cancer patient to further complicate the pharmaceutical treatment regime for their cancer with chronic antidepressant or anxiolytic pharmacologic treatments, many of which work poorly in this patient population and are associated with a significant side effect burden including potential fatalities associated with certain medications (i.e. monoamine oxidase inhibitors). It has also been found that the treatment has a rapid onset following administration of the active agent.

The invention accordingly provides a method of alleviating depression and/or anxiety in a cancer patient, the method comprising administering a single dose of a psychedelic compound which is psilocin or a prodrug of psilocin, or a pharmaceutically acceptable salt thereof, to said cancer patient, wherein: said depression and/or anxiety is alleviated for at least one year; and no further dose of the psychedelic compound is administered to said cancer patient for at least one year after administration of said single dose of the psychedelic compound.

The psychedelic compound has also been found to be effective in reducing existential distress in cancer patients, in particular hopelessness, death anxiety and demoralization (i.e. loss of meaning in life). Accordingly, the invention also provides a method of reducing death anxiety in a cancer patient, the method comprising administering a single dose of a psychedelic compound which is psilocin or a prodrug of psilocin, or a pharmaceutically acceptable salt thereof, to said cancer patient, wherein said death anxiety is reduced for at least one year; and no further dose of the psychedelic compound is administered to said cancer patient for at least one year after administration of said single dose of the psychedelic compound.

The invention further provides a method of reducing demoralization in a cancer patient, the method comprising administering a single dose of a psychedelic compound which is psilocin or a prodrug of psilocin, or a pharmaceutically acceptable salt thereof, to said cancer patient, wherein: said demoralization is reduced for at least one year; and no further dose of the psychedelic compound is administered to said cancer patient for at least one year after administration of said single dose of the psychedelic compound.

The invention yet further provides a method of reducing hopelessness in a cancer patient, the method comprising administering a single dose of a psychedelic compound which is psilocin or a prodrug of psilocin, or a pharmaceutically acceptable salt thereof, to said cancer patient, wherein: said hopelessness is reduced for at least one year; and no further dose of the psychedelic compound is administered to said cancer patient for at least one year after administration of said single dose of the psychedelic compound.

Also provided by the invention is a kit containing a single dose of a psychedelic compound which is psilocin or a prodrug of psilocin, or a pharmaceutically acceptable salt thereof; and instructions for use of said single dose of the psychedelic compound in a method of alleviating depression and/or anxiety in a cancer patient, the method comprising administering a single dose of the psychedelic compound to said cancer patient, wherein: said depression and/or anxiety is alleviated for at least one year; and no further dose of the psychedelic compound is administered to said cancer patient for at least one year after administration of said single dose of the psychedelic compound.

### BRIEF DESCRIPTION OF THE DRAWINGS

FIG. 1 shows the temporal relationships between drug administration, psychosocial interventions and assessments in the initial study. Prep PT: preparatory psychotherapy; 1-day pre-D1: 1 day prior to dose 1; Dose 1: dosing session 1; 1-day post-D1: 1 day after dose 1; Post-integrative PT: post-integrative psychotherapy; 2-wks post-D1: 2 weeks after dose 1; 6-wks post-D1: 6 weeks after dose 1; Safety prep for D2: safety preparation for dosing dose 2; 1-day pre-D2: 1 day prior to dose 2; Dose 2: dosing session 2; 1-day post-D2: 1 day after dose 2; 6-wks post-D2: 6 weeks after dose 2; 26-wks post-D2: 2 weeks after dose 2.

FIG. 2 shows the means (±standard error (SE)) for primary outcome measures for both dose-sequence groups combined at the following time points: Baseline (n=16), 6.5 months (initial study endpoint; n=16), mean 3.2 years (first follow-up; n=15), and mean 4.5 years (second follow-up; n=14). Closed points represent significant within-subject differences relative to the scores at baseline. Longitudinal within-subject effect sizes, represented as Cohen's d, are shown above time points. HADS: Hospital Anxiety and Depression Scale; STAI: State-Trait Anxiety Inventory.

FIG. 3 shows the percentages of participants (in both dose sequence groups combined) fulfilling the criteria for antidepressant or anxiolytic response or antidepressant symptom remission (Hospital Anxiety and Depression Scale-Depression (HADS-D), Beck Depression Inventory (BDI)) at the 4.5-year point (second long-term follow-up; n=14). Clinical response was defined as a 50% or greater decrease in each measure relative to baseline; symptom remission was defined as a 50% or greater decrease in the measure relative to baseline and a score of ≤7 on HADS-D or <12 on BDI.

FIG. 4 shows the means (±standard error (SE)) for secondary outcome measures for participants (in both dose sequence groups combined) at the following time points: Baseline (n=16), 6.5 months (initial study endpoint; n=16), mean 3.2 years (first follow-up; n=15), and mean 4.5 years (second follow-up; n=14). Closed points represent significant within-subject differences relative to scores at baseline.

FIG. 5 shows the percentage of participants who endorsed persisting effects attributable to psilocybin administration on the Persisting Effects Questionnaire at the at the 4.5-year point (second long-term follow-up; n=14): percentage who endorsed “among the top five” or “the single most” personally meaningful experiences; “among the top five” or “the single most” spiritually significant experiences; “moderate,” “strong” or “extreme” positive behavioral change; and “increased moderately” or “increased very much” well-being or life satisfaction.

FIG. 6 shows the scores of HADS-D depression and hopelessness measures assessed two weeks after participants' psilocybin session as a function of elapsed time between the two-week post-psilocybin date and each participant's second long-term follow-up date. Correlation coefficients and p-values are displayed. HADS: Hospital Anxiety and Depression Scale; LTFU: long-term follow-up.

### DETAILED DESCRIPTION OF THE INVENTION

As used herein, the term “about” means any value that the skilled person would appreciate is a reasonable variation of the value that is referred to by the term “about”. Typically, “about” means±10% or ±5%.

The method comprises administering a single dose of a psychedelic compound which is psilocin or a prodrug of psilocin, or a pharmaceutically acceptable salt thereof. The psychedelic compound may accordingly be (i) psilocin or a pharmaceutically acceptable salt of psilocin or (ii) a prodrug of psilocin or a pharmaceutically acceptable salt of the prodrug of psilocin. In one embodiment, the psychedelic compound is a prodrug of psilocin or a pharmaceutically acceptable salt thereof.

A pharmaceutically acceptable salt of a compound is a salt formed with the compound and a non-toxic counter-ion which is suitable for administration to a patient. Pharmaceutically acceptable salts are well-known to the skilled person.

A prodrug of psilocin is a compound which is metabolised (or undergoes a biotransformation) to psilocin after it is administered to the patient. The prodrug of psilocin is typically an ester, carbonate ester, phosphate ester, amide or ether derivative of psilocin. Typically, the prodrug of psilocin is a phosphate ester of psilocin or a pharmaceutically acceptable salt thereof.

In one embodiment, the psychedelic compound is psilocybin or a pharmaceutically acceptable salt thereof. The invention accordingly provides a method of alleviating depression and/or anxiety in a cancer patient, the method comprising administering a single dose of psilocybin or a pharmaceutically acceptable salt thereof to said cancer patient, wherein: said depression and/or anxiety is alleviated for at least one year; and no further dose of psilocybin or the pharmaceutically acceptable salt thereof is administered to said cancer patient for at least one year after administration of said single dose of psilocybin or the pharmaceutically acceptable salt thereof. In one embodiment, the psychedelic compound is psilocybin.

The psychedelic compound may be administered in any suitable form. Typically, the psychedelic compound is administered in a solid form. The psychedelic compound may be in amorphous or crystalline form. The psychedelic compound may be in the form of a solvate. The psychedelic compound may alternatively be administered as a solution.

The psychedelic compound may be administered as a compound which has been synthesised or which has been isolated from a natural source. Alternatively, the psychedelic compound may be administered as part of a natural source which comprises the psychedelic compound. In one embodiment, the method of the invention comprises administering a single dose of a natural source comprising the psychedelic compound. The natural source may be a mushroom of one of the following genera: Psilocybe, Gymnopilus, Panaeolus, Copelandia, Hypholoma, Pluteus, Inocybe, Conocybe, Panaeolina, Gerronema, Agrocybe, Galerina or Mycena. Typically, the natural source may be a mushroom of the Psilocybe genus, for example Psilocybe cubensis, Psilocybe cyanescens, Psilocybe semilanceata or Psilocybe azurescens.

The method is a method of alleviating depression and/or anxiety in a cancer patient. The method may be a method of alleviating depression in a cancer patient. The method may be a method of alleviating anxiety in a cancer patient. As used herein, alleviating depression and/or anxiety includes reducing the symptoms of depression and/or anxiety or achieving remission of depression and/or anxiety. In one embodiment, alleviating depression and/or anxiety comprises reducing the symptoms of depression and/or anxiety. The cancer patient may report a reduction of symptoms of depression and/or anxiety.

In one embodiment, the cancer patient has been identified as being in need of treatment to alleviate depression and/or anxiety. In one embodiment, the cancer patient has indicated that he or she is suffering from depression and/or anxiety.

The symptoms of depression and/or anxiety may be measured using the Hospital Anxiety and Depression Scale (HADS; Zigmond and Snaith (1983), “The hospital anxiety and depression Scale”, Acta Psychiatrica Scand, 67: 361-370). In this test, lower numbers indicate lower levels of depression and/or anxiety. Subscale scores can be calculated for depression (HADS-D) and anxiety (HADS-A). A subscale score equal to or above eight and a full scale score over 12 indicates the possible presence of a clinical disorder.

Accordingly, in the method of the invention a total Hospital Anxiety and Depression Scale score of the cancer patient may be maintained below about 12 for at least one year following the single dose of the psychedelic compound, for instance for at least two years. In one embodiment, the Hospital Anxiety and Depression Scale score of the cancer patient may be maintained below about 10, or below about 8, for at least one year.

The severity of depression may also be measured using the Beck Depression Inventory-II (BDI-II, Beck et al (1988), “Psychometric properties of the Beck Depression Inventory: Twenty-five years of evaluation”, Clin Psych Rev, 8: 77-100). Scores above 12 indicate possible clinical depression.

Accordingly, in the method of the invention a Beck Depression Inventory-II score of the cancer patient may be maintained below about 12 for at least one year following the single dose of the psychedelic compound, for instance for at least two years. In one embodiment, the Beck Depression Inventory-II score of the cancer patient may be maintained below about 10, or below about 8, for at least one year.

The method may further comprise providing the patient with psychotherapy treatment before and/or after the administration of the single dose of the psychedelic compound.

The psychotherapy treatment may be provided before the administration of the single dose of the psychedelic compound, after the administration of the single dose of the psychedelic compound, during the administration of the single dose of the psychedelic compound, or at any combination of two or more of before, during and after the administration of the single dose of psychedelic compound. Typically the psychotherapy treatment may be provided before, during and after the administration of the single dose of the psychedelic compound. As used herein, providing psychotherapy treatment during the administration of the single dose of the psychedelic compound means administering the psychedelic compound during a session of psychotherapy treatment.

The psychotherapy treatment may be provided in the three months before and/or the three months after administration of the single dose of the psychedelic compound. Typically the psychotherapy treatment is provided in the two months before and/or the two months after administration of the single dose of the psychedelic compound. The psychotherapy treatment may be provided in the eight weeks before and/or the eight weeks after administration of the single dose of the psychedelic compound. Where the psychotherapy treatment is provided before and after the administration of the single dose of the psychedelic compound, the psychotherapy treatment before the administration and the psychotherapy treatment after the administration may be provided in different timeframes. For example, the psychotherapy treatment may be provided in the three months before the administration and in the eight weeks after the administration. Alternatively, the psychotherapy treatment before the administration and the psychotherapy treatment after the administration may be provided in the same timeframe.

The cancer patient may receive further psychotherapy in addition to the psychotherapy treatment that may be provided as part of the method of the invention. For example, the cancer patient may receive further psychotherapy at least once a year following the administration of the single dose of the psychedelic compound or the end of the psychotherapy treatment. The cancer patient may receive the further psychotherapy at least twice a year, or at least four times a year.

The psychotherapy treatment may comprise at least 6 hours of psychotherapy treatment. Typically, the psychotherapy treatment comprises at least 8 hours, at least 10 hours, at least 12 hours or at least 20 hours of psychotherapy treatment. Where the psychotherapy treatment is provided before and after the administration of the single dose of the psychedelic compound, the psychotherapy treatment before the administration may comprise the same number of hours as the psychotherapy treatment after the administration. For example, the psychotherapy treatment may comprise 6 hours of psychotherapy treatment before the administration and 6 hours of psychotherapy treatment after the administration. Alternatively, the psychotherapy treatment before the administration may comprise a different number of hours to the psychotherapy treatment after the administration. For example, the psychotherapy treatment may comprise 6 hours of psychotherapy treatment before the administration and 12 hours of psychotherapy treatment after the administration. Where the psychotherapy treatment is provided during the administration of the single dose of the psychedelic compound and before and/or after the administration, the psychotherapy treatment during the administration may comprise the same number of hours as the psychotherapy treatment before and/or after the administration, or the psychotherapy treatment during the administration may comprise a different number of hours as the psychotherapy treatment before and/or after the administration. For example, the psychotherapy treatment may comprise 6 hours of psychotherapy treatment before the administration, 8 hours of psychotherapy treatment during the administration, and 6 hours of psychotherapy treatment after the administration.

The psychotherapy treatment may be provided in sessions at least 30 minutes in duration or at least one hour in duration. Typically, the psychotherapy treatment is provided in sessions at least two hours in duration. The psychotherapy treatment may comprise any number of sessions. Typically the psychotherapy treatment comprises at least 6 sessions. Where the psychotherapy treatment is provided before and after the administration of the single dose of the psychedelic compound, the psychotherapy treatment before the administration may comprise the same number of sessions as the psychotherapy treatment after the administration. For example, the psychotherapy treatment may comprise 3 sessions before and 3 sessions after the administration. Alternatively, the psychotherapy treatment before the administration may comprise a different number of sessions to the psychotherapy treatment after the administration. For example, the psychotherapy treatment may comprise 3 sessions before and 6 sessions after the administration. Any psychotherapy treatment that is provided during the administration of the single dose of the psychedelic compound is typically provided in a single session around 8 hours in duration.

The psychotherapy treatment may be any suitable psychotherapy technique. The psychotherapy treatment may comprise any psychotherapy delivered by two therapists. Typically the psychotherapy treatment is selected from dyadic psychotherapy, supportive psychotherapy, attachment-based psychotherapy, behavioural therapy, body psychotherapy, somatic psychotherapy, brief therapy, cognitive analytical therapy, cognitive behaviour therapy, existential psychotherapy, gestalt therapy, humanistic integrative psychotherapy, hypno-psychotherapy, Jungian analysis, neuro-linguistic psychotherapy, object relations therapy, person-centered psychotherapy, psychodynamic psychotherapy or psychoanalysis, solution-focused brief therapy, transactional analysis, family systems therapy, internal family systems therapy, transpersonal psychotherapy, emotion-focused therapy, compassion-focused therapy, mindfulness-based cognitive therapy. In one embodiment, the psychotherapy treatment is dyadic psychotherapy.

Dyadic psychotherapy is a known technique in psychedelic psychotherapy. As used herein, dyadic psychotherapy is psychotherapy administered by a dyad of two therapists. The dyadic psychotherapy treatment may include elements from supportive psychotherapy; cognitive-behavioral therapy; existentially oriented therapy (i.e. psychotherapies developed specifically by psycho-oncologists to address existentially oriented issues that arise in patients with cancer and especially advanced-staged diagnoses); and psychodynamic/psychoanalytic therapy. In one embodiment, the psychotherapy may be administered by a single therapist.

An advantageous effect of the present invention is that a single dose of the psychedelic compound is useful in achieving long-term alleviation of depression and/or anxiety in cancer patients. Therefore, in the method of the invention the depression and/or anxiety is alleviated for at least one year. Typically, the depression and/or anxiety is alleviated for at least two years, at least three years, at least four years or at least four and a half years. The depression and/or anxiety may be alleviated for at least three years or at least four and a half years. The depression and/or anxiety may be alleviated for at least four and a half years.

The method of the invention may further comprise assessing the cancer patient to determine the extent to which depression and/or anxiety has been alleviated. The cancer patient may be assessed at least one year after the single dose of the psychedelic compound. In one embodiment, the cancer patient is assessed at least two years, at least three years or at least four and a half years after the single dose of the psychedelic compound.

In the method of the invention, no further dose of the psychedelic compound is administered to said cancer patient for at least one year after administration of said single dose of the psychedelic compound. The cancer patient does not therefore receive a therapeutically effective dose of the psychedelic compound in the one year starting the day when the single dose of the psychedelic compound is administered.

In one embodiment, no further of dose of the psychedelic compound is administered to said cancer patient for at least two years, at least three years, at least four years or at least four and a half years after administration of said single dose of the psychedelic compound. The length of time for which no further dose of psilocybin is administered after administration of the single dose of the psychedelic compound may be the same as the length of time for which the depression and/anxiety is alleviated. Alternatively, the length of time for which no further dose of the psychedelic compound is administered after administration of the single dose of the psychedelic compound may be different to the length of time for which the depression and/anxiety is alleviated. For instance, in the method of the invention, the depression and/anxiety may be alleviated for at least two years and no further dose of the psychedelic compound is administered to said cancer patient for at least one year after the single dose, but a further dose of the psychedelic compound may be administered, for instance more than one year after the initial single dose.

As used herein, the term “cancer patient” describes a human patient who has been diagnosed with having a cancer at the time of the administration of the single dose of the psychedelic compound (e.g. psilocybin). The cancer patient may have an active diagnosis of cancer for as long as the depression and/or anxiety in said patient is alleviated. Alternatively, the cancer patient may enter partial or complete remission from cancer during said alleviation of depression and/or anxiety.

In one embodiment, the cancer patient identifies as male. In one embodiment, the cancer patient identifies as female. In one embodiment, the cancer patient identifies as non-binary. The cancer patient may be from 21 to 80 years old. In one embodiment, the cancer patient is from 40 to 60 years old.

The cancer patient may be suffering from stage I, stage II, stage III or stage IV cancer. All stages, but in particular advanced stages, of cancer are associated with elevated rates of psychiatric distress (depression, anxiety) and existential distress. The cancer patient may be suffering from late-stage cancer. Typically, the cancer patient is suffering from stage III or stage IV cancer. Stage I cancer typically refers to cancer where the tumours are small and the cancer is contained with the organ it started in. Stage II cancer typically refers to cancer where the tumours are larger than in stage I, but the cancer has not yet started to spread to surrounding tissues. Stage III cancer typically refers to cancer with large tumours, where the cancer may have spread into the surrounding tissues and nearby lymph nodes. Stage IV cancer typically refers to metastatic cancer, where the cancer has spread from where it started to another organ.

The cancer may be selected from breast cancer, reproductive cancer, lymphoma, leukemia, acute lymphoblastic leukemia (ALL), acute myeloid leukemia (AML), adrenocortical carcinoma, anal cancer, appendix cancer, atypical teratoid/rhabdoid tumor, bile duct cancer, bladder cancer, bone cancer, brain cancer, Burkitt lymphoma, cervical cancer, chronic lymphocytic leukemia (CLL), chronic myelogenous leukemia (CML), chronic myeloproliferative neoplasms, colorectal cancer, cutaneous T-cell lymphoma, esophageal cancer, eye cancer, intraocular melanoma, fallopian tube cancer, gallbladder cancer, gastric (stomach) cancer, gastrointestinal carcinoid tumor, germ cell tumor, hairy cell leukemia, head and neck cancer, heart cancer, Hodgkin lymphoma, intraocular melanoma, islet cell tumor, kidney cancer, Langerhans cell histiocytosis, liver cancer, lung cancer (non-small cell, small cell, pleuropulmonary blastoma, and tracheobronchial tumor), melanoma, mesothelioma, metastatic cancer, mouth cancer, multiple endocrine neoplasia syndrome, multiple myeloma/plasma cell neoplasms, myelodysplastic syndrome, nasopharyngeal cancer, neuroblastoma, non-Hodgkin lymphoma, oral cancer, oropharyngeal cancer, osteosarcoma, ovarian cancer, pancreatic cancer, papillomatosis, paraganglioma, parathyroid cancer, penile cancer, pheochromocytoma, pituitary tumor, primary central nervous system (CNS) lymphoma, primary peritoneal cancer, prostate cancer, rectal cancer, retinoblastoma, salivary gland cancer, sarcoma, skin cancer, small intestine cancer, soft tissue sarcoma, testicular cancer, throat cancer, thymic carcinoma, thyroid cancer, urethral cancer, uterine cancer, and vaginal cancer.

The method of the invention is particularly useful where a cancer patient with said cancer is more likely to have depression and/or anxiety (for example because the cancer has a high mortality rate). Accordingly, the cancer may be selected from breast cancer, reproductive cancer, lymphoma, leukemia, bile duct cancer, brain cancer, esophageal cancer, gallbladder cancer, head and neck cancer, heart cancer, liver cancer, lung cancer, mesothelioma and pancreatic cancer. In one embodiment, the cancer is selected from breast cancer, leukemia, lymphoma, and reproductive cancer.

The cancer patient may have an anxiety-related diagnosis. The anxiety-related diagnosis may include an anxiety-related diagnosis as measured using the Diagnostic and Statistical Manual of Mental Disorders (DSM-IV). The anxiety-related diagnosis may be selected from an adjustment disorder, panic disorder, post-traumatic stress disorder, obsessive-compulsive disorder, social phobia, acute stress disorder, and generalized anxiety disorder. Typically, the anxiety-related diagnosis is an adjustment disorder or generalized anxiety disorder.

The method comprises administering a single dose of the psychedelic compound. In one embodiment, a single dosage form (such as a tablet) comprising the psychedelic compound is administered to the cancer patient in order to provide the single dose. However, as this skilled person will appreciate, this does not necessarily mean that only a single dosage form comprising the psychedelic compound may be administered. The single dose of the psychedelic compound may be administered as two or more separate dosage forms, which two or more separate dosage forms are administered simultaneously or shortly after one another (for instance less than 2 hours apart). The single dose may also be administered over a period of time, for example if being administered as an infusion. The single dose of the psychedelic compound is typically administered in a timeframe of no greater than about one hour.

The psychedelic compound is administered in an amount effective to alleviate depression and/or anxiety for at least one year. An effective amount of the psychedelic compound may be from about 0.001 mg/kg to about 10 mg/kg, for instance from about 0.01 mg/kg to about 1 mg/kg, where mg/kg is mg per kg of the patient's body weight at the time of the administration of the single dose. Typically, an effective amount of the psychedelic compound may be a dose of from about 0.1 mg/kg to about 0.5 mg/kg, or from about 0.2 mg/kg to about 0.4 mg/kg. In one embodiment, the effective amount of the psychedelic compound is about 0.3 mg/kg. In the method of the invention, the effective amount of the psychedelic compound is administered as a single dose.

The single dose of the psychedelic compound may be from about 0.1 to about 100 mg. In one embodiment, the psychedelic compound is administered in a single effective dose of from about 10 mg to about 40 mg. Typically, the single dose may be from about 10 mg to about 35 mg, or from about 15 mg to about 30 mg, or from about 20 mg to about 30 mg. In one embodiment, the single dose is about 25 mg. In one embodiment, the single dose is from about 1 mg to about 10 mg.

In one embodiment, the psychedelic compound is psilocybin. The method of the invention may accordingly comprise administering a single dose of psilocybin which is from about 0.01 mg/kg to about 1 mg/kg. The single dose of psilocybin may be from about 0.1 mg/kg to about 0.5 mg/kg, or from about 0.2 mg/kg to about 0.4 mg/kg. In one embodiment, the single dose of psilocybin is about 0.3 mg/kg.

Alternatively, the method of the invention may comprise administering a single dose of psilocybin which is from about 10 mg to about 40 mg. The single dose of psilocybin may be from about 10 mg to about 35 mg, or from about 15 mg to about 30 mg, or from about 20 mg to about 30 mg. In one embodiment, the single dose of psilocybin is about 25 mg.

In one embodiment, the cancer patient is not administered a separate anxiolytic or antidepressant for the period during which anxiety and/or depression is alleviated.

A single dose of the psychedelic compound has also been shown to be effective in alleviating existential distress in cancer patients for at least one year. As used herein, alleviating existential distress includes reducing levels of at least one of death anxiety, hopelessness and demoralization. Typically, alleviating existential distress includes reducing levels of hopelessness, death anxiety and/or demoralization.

In the method of reducing death anxiety in a cancer patient, death anxiety is reduced relative to the death anxiety of the patient before the administration of the single dose of the psychedelic compound. Death anxiety is measured according to the Death Anxiety Scale (Templer (1970), “The construction and validation of a death anxiety scale”, J Gen Psychol, 82: 165-177). Scores below 8 are considered normative levels of death anxiety. Accordingly, in the method of the invention a death anxiety score of less than 8 may be maintained for at least one year.

In the method of the invention the death anxiety is reduced for at least one year. Typically, the death anxiety is reduced for at least two years, at least three years, at least four years or at least four and a half years. The death anxiety may be reduced for at least three years or at least four and a half years. The death anxiety may be reduced for at least four and a half years.

In the method of the invention, no further dose of the psychedelic compound is administered to said cancer patient for at least one year after administration of said single dose of the psychedelic compound. Typically, no further of dose of the psychedelic compound is administered to said cancer patient for at least two years, at least three years, at least four years or at least four and a half years after administration of said single dose of the psychedelic compound. The length of time for which no further dose of the psychedelic compound is administered after administration of the single dose of the psychedelic compound may be the same as the length of time for which the death anxiety is reduced. Alternatively, the length of time for which no further dose of the psychedelic compound is administered after administration of the single dose of the psychedelic compound may be different to the length of time for which the death anxiety is reduced.

The invention also relates to a method of reducing demoralization in a cancer patient. In the method, demoralization is reduced relative to the demoralization of the patient before the administration of the single dose of the psychedelic compound. Demoralization is measured according to the Demoralization Scale (Kissane et al. (2004), “The demoralization scale: A report of its development and preliminary validation”, J Palliat Care, 20: 269-276). Scores above 30 are considered indicative of clinical levels of demoralization. Accordingly, in the method of the invention demoralization score of less than 30 may be maintained for at least one year.

In the method of the invention the demoralization is reduced for at least one year. Typically, the demoralization is reduced for at least two years, at least three years, at least four years or at least four and a half years. The demoralization may be reduced for at least three years or at least four and a half years. The demoralization may be reduced for at least four and a half years.

In the method of the invention, no further dose of the psychedelic compound is administered to said cancer patient for at least one year after administration of said single dose of the psychedelic compound. Typically, no further of dose of the psychedelic compound is administered to said cancer patient for at least two years, at least three years, at least four years or at least four and a half years after administration of said single dose of the psychedelic compound. The length of time for which no further dose of the psychedelic compound is administered after administration of the single dose of the psychedelic compound may be the same as the length of time for which the demoralization is reduced. Alternatively, the length of time for which no further dose of the psychedelic compound is administered after administration of the single dose of the psychedelic compound may be different to the length of time for which the demoralization is reduced.

The invention also relates to a method of reducing hopelessness in a cancer patient. In the method, hopelessness is reduced relative to the hopelessness of the patient before the administration of the single dose of the psychedelic compound. Hopelessness is measured according to the Hopelessness Assessment in Illness instrument (Rosenfeld et al. (2011), “Assessing hopelessness in terminally ill cancer patients: Development of the Hopelessness Assessment in Illness Questionnaire”, Psychol Assess, 23: 325-336), on a scale of 0-16. Higher scores indicate higher levels of hopelessness. Accordingly, in the method of the invention a Hopelessness Assessment in Illness score of less than 8 may be maintained for at least one year.

In the method of the invention the hopelessness is reduced for at least one year. Typically, the hopelessness is reduced for at least two years, at least three years, at least four years or at least four and a half years. The hopelessness may be reduced for at least three years or at least four and a half years. The hopelessness may be reduced for at least four and a half years.

In the method of the invention, no further dose of the psychedelic compound is administered to said cancer patient for at least one year after administration of said single dose of the psychedelic compound. Typically, no further of dose of the psychedelic compound is administered to said cancer patient for at least two years, at least three years, at least four years or at least four and a half years after administration of said single dose of the psychedelic compound. The length of time for which no further dose of the psychedelic compound is administered after administration of the single dose of the psychedelic compound may be the same as the length of time for which the hopelessness is reduced. Alternatively, the length of time for which no further dose of the psychedelic compound is administered after administration of the single dose of the psychedelic compound may be different to the length of time for which the hopelessness is reduced.

The method of reducing death anxiety, the method of reducing demoralization and the method of reducing hopelessness may be as defined above for the method of alleviating anxiety and/or depression.

### Kits

The kit of the invention contains a single dose of the psychedelic compound; and instructions for use of said single dose of the psychedelic compound in the method of alleviating depression and/or anxiety in a cancer patient. Said instructions may include, for example, instructions for psychotherapy treatment to be provided before, during, and/or after the administration of the single dose of the psychedelic compound. The single dose of the psychedelic compound is typically in the form of a pharmaceutical formulation, for instance as described below.

The invention also provides a kit comprising a single dose of the psychedelic compound and instructions for use of said single dose of the psychedelic compound in a method of reducing death anxiety, hopelessness or demoralization in a cancer patient according to the invention.

### Formulation of the Psychedelic Compound

The psychedelic compound may be administered to the cancer patient by any acceptable route of administration including, but not limited to, inhaled, oral, nasal, topical (including transdermal) and parenteral modes of administration. The psychedelic compound may be administered as, for example: a tablet, capsule, powder, solution or suspension for oral administration; a solution or suspension for injection; or a solution, suspension or powder for inhalation. The psychedelic compound may be administered in a food stuff, for instance a food stuff comprising a natural source of the psychedelic compound such as a fungus of the genus Psilocybe.

Depending on the mode of administration used, the psychedelic compound (e.g. psilocybin) may be formulated with an appropriate conventional carrier, excipient or diluent. Pharmaceutically acceptable excipients, carriers and diluents are well known to the skilled person.

The diluent may be any pharmaceutically acceptable diluent. The diluent is typically suitable for parenteral administration or for oral administration. Examples of suitable liquid diluents include water, ethanol and glycerol. The diluent may alternatively be selected from solid diluents such as lactose, dextrose, saccharose, cellulose, corn starch and potato starch. The diluent may contain buffer components to control the pH. The buffers may be derived from phosphate, citrate or acetate. The diluent may also contain sodium chloride.

The excipient may be selected from: lubricants, e.g. silica, talc, stearic acid, magnesium or calcium stearate, and/or polyethylene glycols; binding agents, e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose, glycerine, mannitol or sorbitol.

Suspensions or emulsions may contain as carrier, for example a natural gum, agar, sodium alginate, pectin, methylcellulose, carboxymethylcellulose, or polyvinyl alcohol. The suspension or solutions for intramuscular injections may contain, together with the psychedelic compound, a pharmaceutically acceptable carrier, e.g. sterile water, olive oil, ethyl oleate, glycols, e.g. propylene glycol, and if desired, a suitable amount of lidocaine hydrochloride. Solutions for injection or infusion, or for inhalation, may contain as carrier, for example, sterile water or they may be in the form of sterile, aqueous, isotonic saline solutions.

The invention is described in more detail by the following Example. Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

The texts of references cited in this disclosure are herein incorporated by reference in their entireties.

### Example

The present example assesses the long-term effects of a single dose of psilocybin (0.3 mg/kg) in conjunction with psychotherapy in patients with cancer-related psychiatric and existential distress.

### Methods

29 participants were randomly assigned to one of two groups: psilocybin (0.3 mg/kg) on the first medication session followed by niacin (250 mg) on the second session (i.e. psilocybin-first group), or niacin (250 mg) on the first medication session followed by psilocybin (0.3 mg/kg) on the second session (i.e. niacin-first group). Psilocybin and niacin were administered in identically appearing gelatin capsules.

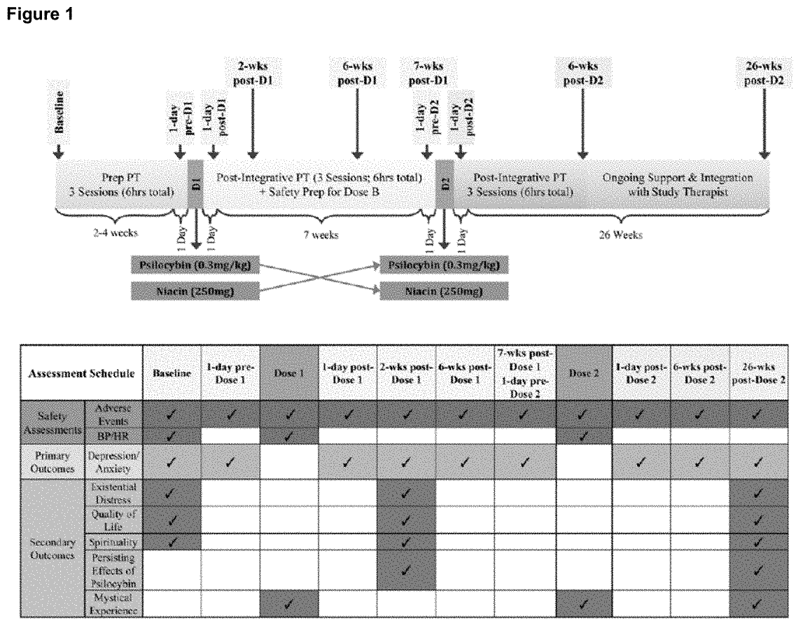

Participants received three 2-hour preparatory psychotherapy sessions in order to review the purpose and intention of participation in the study, treatment goals, and structure of the treatment sessions. Medications were administered in 8-hour treatment sessions which included a discussion of the participant's subjective experience with the treatment team to consolidate the memory of the experience and to begin the process of post-integrative psychotherapy. Participants also received three 2-hour post-medication integration sessions (repeated after the second dosing session) to further consolidate the memory of the experience and to continue the process of psychological integration. All psychotherapy treatment sessions were delivered by a dyadic therapy team trained in a blend of psychotherapies with an evidence base to support their use in cancer patients (drawing upon principles of palliative care therapy, existential psychotherapy, cognitive-behavioural therapy, psychoanalysis therapy, and transpersonal psychology) and were taught about the safety and clinical pharmacology of psilocybin. Each therapy dyad underwent six confidential one-to-one sessions to build their alliance and exchange ideas about psychedelic therapy. The trial employed a crossover design at seven weeks following the first drug administration, with an outcome assessment at 6.5 months (26 weeks) following the second drug administration (i.e. after the crossover). An overview of this part of the trial is shown in FIG. 1.

Two long-term follow ups (LTFUs) were carried out to assess the continued effect of psilocybin. Of the original 29 participants, all 16 participants who were not deceased at the time of LTFU were contacted (the remaining 13 participants were deceased). Of the 16 participants who were contacted, 15 agreed to participate in the LTFU and completed measures through a secure online portal. One participant died (from cancer-related complications) after completing the first LTFU and prior to the second LTFU, leaving us with 14 participants at the second LTFU. The first and second LTFUs occurred on average 3.2 years (range 2.3-4.5 years) and 4.5 years (range 3.5-5.5 years) following the participants' psilocybin dosing date, respectively.

### Participants

Demographic information of the participants assessed in the LTFUs is presented in Table 1. At the first LTFU, the mean age of participants was 53 years old (standard deviation (SD)=16 years), and they were predominantly female (60%). The majority was non-Hispanic White (93%), followed by Asian (6%). Forty percent reported Catholic/Christian or Jewish beliefs, and one-third (33%) reported atheist/agnostic beliefs, followed by “other” faith/tradition (13%). Gynecological cancers (33%) comprised the majority of disease sites, followed by breast (20%) and lymphomas (20%). Slightly more than half (60%) were diagnosed with early stage (I-II) cancers versus later stage (III-IV, 53%) at the parent study end point. Of note, at the second LTFU, 71% of participants had reportedly entered partial or complete remission from their cancers, and 29% were in the active stages of their diseases.

TABLE 1
Demographic and clinical characteristics
of study participants at LTFU follow-ups.

| | | Total | |
|---|---|---|---|
| Characteristic | Categories | N = 15 | |
| Sex | Female | 9 | 60.00% |
| | Male | 6 | 40.00% |
| Age at follow- | Range 25-73 | 53 (15.5) | |
| up; mean (SD) | | | |
| Race | White/Caucasian | 14 | 93.33% |
| | Asian | 1 | 6.67% |
| Religious/ | Atheist/agnostic | 5 | 33.33% |
| spiritual beliefs | Jewish | 3 | 20.00% |
| | Catholic | 1 | 6.67% |
| | Other Christian | 2 | 13.33% |
| | Other faith/tradition | 2 | 13.33% |
| Site of cancer | Breast | 3 | 20.00% |
| | Reproductive | 5 | 33.32% |
| | Lymphoma/leukemia | 3 | 20.00% |
| | Other types | 4 | 26.67% |
| Stage of cancer | Stage IV | 2 | 13.33% |
| | Stage III | 4 | 26.67% |
| | Stage II | 3 | 20.00% |
| | Stage I | 5 | 33.32% |
| | Other | 1 | 6.67% |
| SCID | Adjustment disorder w/ | 2 | 13.33% |
| (DSM-IV-TR) | anxiety and depressed | | |
| diagnosis | mood, chronic | | |
| | Adjustment disorder w/ | 12 | 80.00% |
| | anxiety, chronic | | |
| | Generalized anxiety | 1 | 6.67% |
| | disorder | | |
| Hallucinogen | No | 8 | 53.33% |
| use | Yes | 7 | 46.67% |
| Education | Part-college | 2 | 13.33% |
| | Graduated 4-year college | 4 | 26.67% |
| | Completed grad/professional | 9 | 60.00% |
| | school | | |

DSM-IV-TR: Diagnostic and Statistical Manual of Mental Disorders; SD: standard deviation; SCID: Structured Clinical Interview for DSM Disorders.

The majority of participants (93%) met Diagnostic and Statistical Manual of Mental Disorders (DSM-IV-TR, American Psychiatric Association, 2000) criteria for cancer-related adjustment disorder with anxious and/or depressed features, followed by generalized anxiety disorder (7%). Compared to the 29 participants originally involved, the proportions of participants in the LTFU study were roughly equivalent in all demographic variables with the exception of cancer type. There was a greater proportion of reproductive cancer in this LTFU sample, and no participants carried a diagnosis of digestive cancers in the LTFU sample (compared to 21% in the original sample).

### Psychiatric Interventions Received During Follow-Up Period

A total of 13 out of the 14 participants who completed the second LTFU time point provided information regarding their use of psychotherapy or pharmacological interventions after completion of the parent study. One participant who participated in the LTFU passed away prior to the administration of this assessment. Participants provided the name, dosage, duration, and reason for medication prescription, as well as type, duration, and reason for any psychotherapy intervention received during the LTFU period.

During the LTFU period five participants (39%) reported receiving some form of psychotherapy since completion of the parent trial, with one (8%) receiving psychotherapy specifically targeting cancer-related psychological distress. Three participants (23%) received some form of psychotropic drug treatment, with no participants receiving psychotropic medication specifically targeting cancer-related psychological distress during the LTFU period. None of the participants reported lasting negative or adverse effects from the psilocybin-assisted therapy experiences.

### Outcome Measures

Primary outcomes (anxiety and depression) were measured according to the following methods:
Hospital Anxiety and Depression Scale (HADS) (Zigmond and Snaith, 1983)—this is widely used in hospital settings to screen for the severity of anxiety and depression. It contains 14 questions rated on a four-point scale (total score (HAD-T) ranges from 0-56). Subscale scores can be calculated for depression (HADS-D) and anxiety (HADS-A). Although there is no single accepted cut-off score, it is suggested that subscale scores equal to or above eight and full-scale scores over 12 indicate the possible presence of a clinical disorder (Snaith and Zigmond, 1994). The HADS has shown good reliability (Cronbach's a ranging from 0.80-0.93) and has been well validated (Herrmann, 1997).
Beck Depression Inventory-II (BDI-II) (Beck et al., 1988)—a widely used self-report screening measure for depression. The BDI-II consists of 21 questions about depressive symptoms experienced over the past two weeks rated on a three-point scale (total score ranges from 0-63). Scores above 12 indicate possible clinical depression. This measure has shown good reliability (internal consistency of 0.90) and factorial validity (Storch et al. 2004).
Spielberger State-Trait Anxiety Inventory (STAI) (Spielberger, 1983)—a well-known measure of anxiety consisting of scales for state (STAI-S) and trait-level anxiety (STAI-T). Each scale contains 20 items rated on a four-point scale (subscale scores ranging from 20-80). Scores above 40 on each subscale indicate clinical presence of anxiety symptoms. The measure has shown good reliability (Cronbach's a ranging from 0.83-0.86) and discriminant validity (Quek et al., 2004).

Secondary outcomes were measured as follows:

### (i) Existential Distress

Cancer-related existential distress (demoralization, hopelessness, attitudes and effects associated with disease progression and death) was assessed using the following methods:
The Death Anxiety Scale (DAS) (Templer, 1970)—a 15-item measure that has been used most frequently to assess death anxiety. Items are scored “true” and “false” and then scored as one and zero, respectively. Total scores range between 0-15. Higher scores represent increased severity of death anxiety. Scores below eight are considered normative levels of death anxiety. Templer (1970) reported adequate test-retest reliability (r=0.83) and validity.
The Hopelessness Assessment in Illness (HAI) (Rosenfeld et al., 2011)—an eight-item instrument developed for use in patients with advanced cancer. Total scores range from 0-16. Higher scores indicate higher levels of hopelessness. Data have not been published regarding recommended clinical cut-off scores for this measure. This measure has shown adequate internal consistency (Cronbach's α=0.87) and concurrent validity (r=0.70-0.78; Rosenfeld et al., 2011).
The Demoralization Scale (DS) (Kissane et al., 2004)—a 24-item questionnaire measuring existential distress encompassing five factors. These dimensions include loss of meaning, despair, disheartened feelings, helpless feelings, and a sense of failure. Likert scale items range from 0-4, and total scores range from 0-96. Score above 30 are considered indicative of clinical levels of demoralization. This measure has shown good reliability (Cronbach's a ranging from 0.71-0.89) and concurrent validity, with regard to related scales (Kissane et al. 2004).

### (ii) Spirituality

Spirituality was assessed using the Functional Assessment of Chronic Illness Therapy-Spiritual Well-Being (FACIT-Sp-12; Bredle et al., 2011). This is a 12-item measure of spiritual well-being among individuals with cancer and other forms of chronic illness. Items are rated on a five-point Likert scale. The measure yields three subscales: a sense of meaning/peace in life, a sense of comfort from one's faith, and total spiritual well-being score. Total scores for each subscale range from 0-32, 0-16, and 0-48, respectively. Data have not been published regarding recommended cutoff scores for this measure. This measure has shown good reliability (Cronbach's a ranging from 0.81-0.88) and has been well validated (Bredle et al. 2011).

(iii) Persisting Effects of Psilocybin

Persisting effects of psilocybin were assessed using the Persisting Effects Questionnaire (Griffiths et al., 2006, 2008) assesses self-rated changes in one's attitude, mood, behavior, and experience of spirituality. This measure can detect longitudinal effects of psilocybin administration. An 89-item version was administered to participants in the original study.

In the present LTFU study, the following four questions were drawn from the original version. Participants were asked to indicate: (a) the personal meaningfulness of the psilocybin experience (rated from 1-8, with 1=no more than routine everyday experiences; 7=among the five most meaningful experiences of my life; and 8=the single most meaningful experience of my life); (b) the degree to which the experience was spiritually significant (rated from 1-6, with 1=not at all; 5=among the five most spiritually significant experiences of my life; 6=the single most spiritually significant experience of my life); (c) whether the experience and their contemplation of that experience led to changes in their current sense of personal well-being or life satisfaction (rated from +3=increased very much; +2=increased moderately; +1=increased slightly, 0=no change, −1=decreased slightly, −2=decreased moderately, and −3=decreased very much; (d) and the degree to which their behaviors have changed positively as a result of the experience (rated from 0=none, 1=so slight cannot decide, 2=slight, 3=moderate, 4=strong, and 5=extreme).

### (iv) Mystical Experience

Mystical experience was assessed using a Mystical Experience Questionnaire (MEQ-30; MacLean et al., 2012), a 30-item self-report questionnaire that measures qualities of mystical-type experiences occasioned by a psychedelic. The scale comprises four subscales: “Mystical” factor, “Transcendence of time and space,” “Positive mood,” and “Ineffability.” Items are measured on a six-point Likert scale ranging from zero (not at all) to six (extremely, more than any other time in my life). Total scores range from 30-180. This measure has shown good reliability (Cronbach's a ranging from 0.80-0.93) and has been well validated (MacLean et al. 2012).

### Data Analysis

Regarding all analyses for both the primary and secondary outcome assessments, both the psilocybin-first and niacin-first dose sequence groups were collapsed and combined into one group. Reasons for this decision included the crossover design, which prevented valid between-group comparisons subsequent to the crossover, and a need to increase power given the modest sample size.

The long-terms effects of psilocybin on variables of interest were evaluated using four repeated measures regressions, estimated within the mixed effect repeated measurement (MMRM) model. Planned within-subject t-tests (Tukey's post-hoc) were conducted comparing scores at baseline to the following time points for primary and secondary outcomes: 6.5 months after the second medication session, and the first and second LTFU points. Planned within subject t-tests were also conducted comparing scores at parent study endpoint (6.5 months) to the two LTFU points. Remissions status (partial or complete remission versus an active diagnosis of cancer) was entered as a covariate into the MMRM model to examine whether it significantly impacted symptomatology on primary and secondary outcome measures.

Rates of clinically significant responses and symptom remission were calculated for primary outcome measures that have empirical support in defining antidepressant (HADS-D, BDI) or anxiolytic response (HADS-A) for each of the dose-sequence groups. Clinical significance was defined as 50% or greater reduction in a score at a particular assessment point relative to baseline. Antidepressant symptom remission was defined as 50% or greater reduction in depressive symptoms in addition to HADS-D≤7 (Hung et al., 2012) or BDI≤12 (Reeves et al., 2012; Riedel et al., 2010).

Participants were asked to reflect on their psilocybin session and to rate persisting effects attributed to the medication sessions on four items on the Persisting Effects Questionnaire at the second LTFU: positive behavioral change, meaningfulness, spiritual significance, and increases in personal well-being. Ratings of these persisting effects were expressed as proportions. Spearman rank correlation coefficients (for use in nonparametric tests) were calculated between total scores on the MEQ-30 assessed at the end of their psilocybin session days and change scores on primary and secondary measures between baseline and the second LTFU assessment.

To determine whether length of time between participants' psilocybin session and the LTFU predicted long-term clinical change, Spearman rank correlation coefficients were calculated between change scores on measures of anxiety, depression, and existential distress (i.e. second LTFU subtracted from two-weeks post-psilocybin) and the total number of days elapsed between each participant's unique individual psilocybin session and the date of their second LTFU assessment. Of note, the range of two-weeks post-psilocybin dose in comparison to the final long-term outcome was selected because the two-week post-dose assessment was the first LTFU point that included all primary and secondary measures.

### Results

### (i) Primary Outcomes

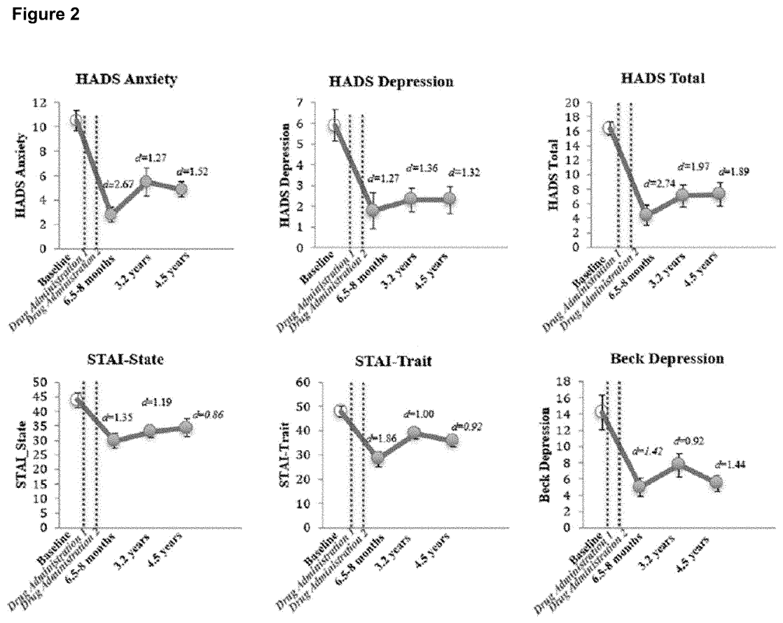

Statistically significant reductions relative to baseline on all of the primary measures measuring anxiety and depression were observed at each of the 6.5-month, first and second LTFU points (see FIG. 2 and Table 2). This represented large, statistically significant reductions in symptoms since baseline at the 6.5-month point (mean Cohen's d=1.90, range 1.27-2.67), first LTFU (mean Cohen's d=1.30, range 0.93-1.97), and second LTFU point (mean Cohen's d=1.41, range 0.86-1.89).

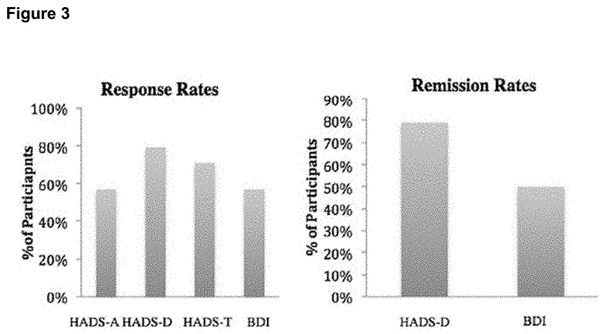

At the second LTFU point, 57% of participants showed a clinically significant anxiolytic response on the HADS-A. Seventy-one percent of participants reported clinically significant reductions in global psychological distress on the HADS-T, measuring anxiety and depression combined. Lastly, percentages of clinical responses for depression on the HADS-D and BDI ranged from 57-79%, and depression symptom remission rates ranged from 50-79% at the second LTFU (see FIG. 3).

TABLE 2
Participant ratings on primary and secondary questionnaires.

| | Assessment time point | | | | | | | |
|---|---|---|---|---|---|---|---|---|
| Measure | Baseline | | 6.5-8 months | | 3.2 years | | 4.5 years | |
| HADS Anxiety | 10.56 | (0.93) | 2.81 | (0.95)^{a} | 5.50 | (0.93)^{a} | 4.99 | (0.98)^{a} |
| HADS Depression | 5.88 | (0.71) | 1.75 | (0.73)^{a} | 2.25 | (0.71)^{a} | 2.30 | (0.75)^{b} |
| HADS Total | 16.45 | (1.32) | 4.38 | (1.35)^{a} | 7.13 | (1.32)^{a} | 7.34 | (1.39)^{a} |
| STAI State Anxiety | 43.94 | (2.51) | 29.84 | (2.58)^{a} | 33.00 | (2.51)^{b} | 34.41 | (2.67)^{c} |
| STAI Trait Anxiety | 47.81 | (2.24) | 28.23 | (2.75)^{a} | 3.84 | (2.85)^{c} | 35.78 | (3.02)^{b} |
| Beck Depression | 14.19 | (1.49) | 5.09 | (1.54)^{a} | 7.75 | (1.49)^{b} | 5.45 | (1.59)^{a} |
| Demoralization | 31.88 | (2.61) | 16.84 | (2.67)^{a} | 13.29 | (2.69)^{a} | 14.32 | (2.76)^{a} |
| Hopelessness | 5.75 | (0.51) | 1.65 | (0.52)^{a} | 2.29 | (0.52)^{a} | 1.65 | (0.54)^{a} |
| Death anxiety | 8.06 | (0.78) | 6.09 | (0.79)^{b} | 5.68 | (0.79)^{b} | 5.75 | (0.81)^{c} |
| Meaning/peace | 19.43 | (0.92) | 26.34 | (0.95)^{a} | 19.27 | (0.95) | 20.20 | (0.98) |
| Faith | 6.75 | (1.32) | 9.77 | (1.34)^{b} | 9.31 | (1.35)^{c} | 10.43 | (1.37)^{b} |
| Spiritual well-being | 55.69 | (3.16) | 70.58 | (3.12)^{a} | 59.85 | (3.24) | 65.04 | (3.31)^{c} |
| Social relationships | 13.91 | (0.75) | 15.27 | (0.76) | 15.54 | (0.77) | 14.67 | (0.79) |
| Environmental health | 15.50 | (0.53) | 16.54 | (0.53)^{c} | 16.72 | (0.54)^{c} | 17.00 | (0.55)^{c} |
| Physical health | 15.00 | (0.69) | 16.35 | (0.70)^{c} | 16.33 | (0.71) | 13.89 | (0.73) |
| Psychological health | 13.58 | (0.43) | 15.70 | (0.45)^{a} | 15.48 | (0.45)^{b} | 14.80 | (0.46) |

HADS: Hospital Anxiety and Depression Scale; SD: standard deviation; STAI: State-Trait Anxiety Inventory.
Data are means (SDs) collapsed across both dose sequence groups (N = 16, N = 15, N = 15, N = 14 at baseline, 6.5 months, mean 3.2 years and mean 4.5 years, respectively).
Superscripts indicate significant within-subject differences from baseline to time point (^{a}p < 0.001, ^{b}p < 0.01, ^{c}p < 0.05).

### (ii) Secondary Outcomes

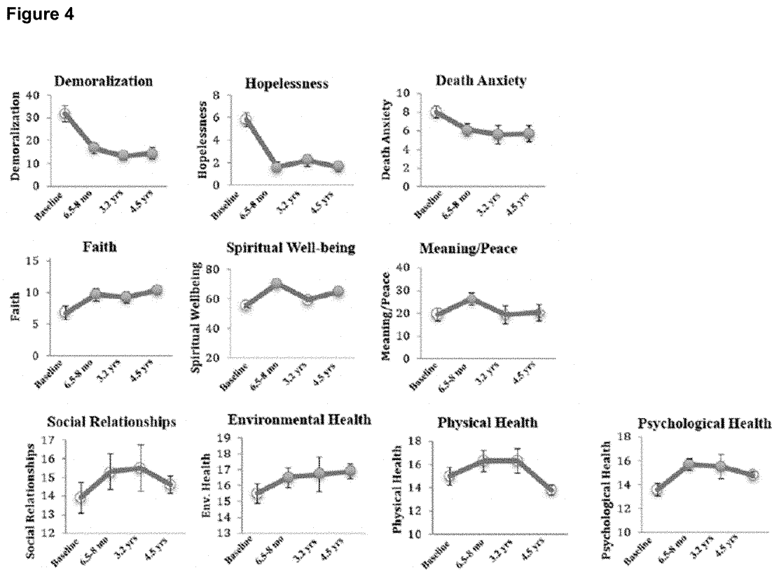

There were significant reductions in hopelessness, demoralization and death anxiety at the 6.5-month, first and second LTFU points relative to baseline. These represented large, statistically significant reductions in symptoms since baseline at the 6.5-month point (mean Cohen's d=1.39, range 0.88-2.00), first LTFU (mean Cohen's d=1.39, range 0.80-1.77), and second LTFU (mean Cohen's d=1.60, range 1.00-2.00). Results are presented in FIG. 4.

There were also significant improvements in spiritual well-being and faith domains (FACIT-Sp-12) at the second LTFU relative to baseline.

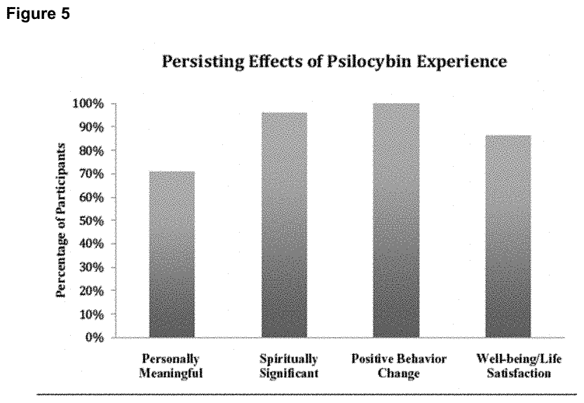

Participant ratings of persisting effects are displayed in FIG. 5. Participants indicated positive attributions to the psilocybin experience that persisted until the second LTFU. Seventy-one percent of participants continued to rate the psilocybin experience the single or top-five most personally meaningful experience(s) of their lives, while ninety-six percent rated the psilocybin experience the single or top-five most spiritually significant experience(s) of their lives. Participants appraised the psilocybin session as increasing life satisfaction or wellbeing at a rate of 86%. Lastly, 100% of volunteers reported “moderate,” “strong” or “extreme” positive behavioral change attributed to the psilocybin experience. Cancer remission status (partial or complete remission versus an active diagnosis of cancer) did not significantly interact with any of the scores on primary or secondary outcome measures.

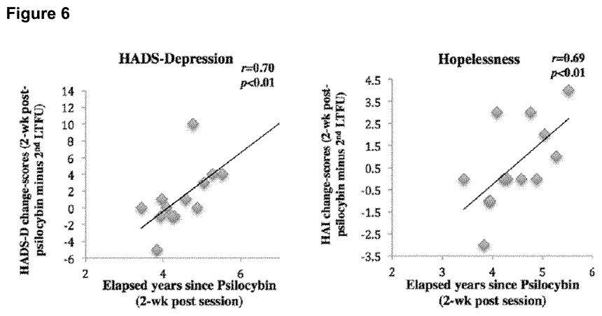

The length of time between psilocybin session and follow-up (i.e. second LTFU relative to each participant's two-week-post-psilocybin dosing date) correlated positively with depression and hopelessness change scores (second LTFU subtracted from two-weeks post-psilocybin dose scores) on the following measures: HAD-D (r=0.70, p<0.01) and HAI (r=0.69, p<0.01). Results are depicted in FIG. 6.

Participants were asked open-ended questions about their psilocybin-assisted psychotherapy experience to further understand the enduring high ratings of persisting effects at the second LTFU.

### CONCLUSION

Psilocybin treatment is shown to be associated with long-term and significant reductions in anxiety, depression, hopelessness, demoralization and death anxiety. Significant clinical improvements are observed up to 4.5 years after psilocybin administration. In particular, 60-80% of participants met criteria for clinical antidepressant or anxiolytic response and remission not only at 6.5 months after psilocybin administration, but also at the 4.5 year time point.

A greater amount of time between participants' psilocybin session and the second LTFU resulted in stronger reductions in reports of depression and hopelessness. This suggests that certain domains of cancer-related distress could continue to improve over time in relation to a single psilocybin session.

### REFERENCES

American Psychiatric Association (2000), Diagnostic and Statistical Manual of Mental Disorders: DSM-IV-TR. Washington, D.C.: American Psychiatric Association.
Beck et al (1988), “Psychometric properties of the Beck Depression Inventory: Twenty-five years of evaluation”, Clin Psych Rev, 8: 77-100.
Bredle et al. (2011), “Spiritual well-being as a component of health-related quality of life: The functional assessment of chronic illness therapy—spiritual well-being scale (FACIT-Sp)”, Religions, 2: 77-94.
Griffiths et al. (2008), “Mystical-type experiences occasioned by psilocybin mediate the attribution of personal meaning and spiritual significance 14 months later”, J Psychopharmacol, 22: 621-632.
Griffiths et al. (2006), “Psilocybin can occasion mystical-type experiences having substantial and sustained personal meaning and spiritual significance”, Psychopharmacology (Berl), 187: 268-283.
Herrmann (1997), “International experiences with the Hospital Anxiety and Depression Scale: A review of validation data and clinical results”, J Psychosom Res, 42: 17-41
Hung et al. (2012), “The cut-off points of the Depression and Somatic Symptoms Scale and the Hospital Anxiety and Depression Scale in detecting non-full remission and a current major depressive episode”, Int J Psychiatry Clin Pract, 16: 33-40.
Kissane et al. (2004), “The demoralization scale: A report of its development and preliminary validation”, J Palliat Care, 20: 269-276.
MacLean et al. (2012), “Factor analysis of the mystical experience questionnaire: A study of experiences occasioned by the hallucinogen psilocybin”, J Sci Study Relig, 51: 721-737.
Quek et al. (2004), “Reliability and validity of the Spielberger State-Trait Anxiety Inventory (STAI) among urological patients: A Malaysian study”, Med J Malaysia, 59: 258-267.
Reeves et al. (2012), “Calibration of response and remission cut-points on the Beck Depression Inventory-Second Edition for monitoring seasonal affective disorder treatment outcomes”, J Affect Disord, 138: 123-127.
Riedel et al. (2010), “Response and remission criteria in major depression—a validation of current practice”, J Psychiatr Res, 44: 1063-1068.
Rosenfeld et al (2011), “Assessing hopelessness in terminally ill cancer patients: Development of the Hopelessness Assessment in Illness Questionnaire”, Psychol Assess, 23: 325-336.
Ross S et al. (2016), “Rapid and sustained symptom reduction following psilocybin treatment for anxiety and depression in patients with life-threatening cancer: A randomized controlled trial”, J Psychopharmacol, 30: 1165-1180.
Snaith and Zigmond (1994), The Hospital Anxiety and Depression Scale with the irritability-depression-anxiety scale and the Leeds Situational Anxiety Scale: Manual. London: GL Assessment Ltd.
Spielberger (1983), Manual for the State-Trait Anxiety Inventory (Self-Evaluation Questionnaire), Palo Alto: Consulting Psychologists Press, Inc.
Storch et al (2004), “Factor structure, concurrent validity, and internal consistency of the beck depression inventory—second edition in a sample of college students”, Depress Anxiety, 19: 187-189.
Templer (1970), “The construction and validation of a death anxiety scale”, J Gen Psychol, 82: 165-177.
Zigmond and Snaith (1983), “The hospital anxiety and depression Scale”, Acta Psychiatrica Scand, 67: 361-370

1. A method of alleviating depression and/or anxiety in a cancer patient, the method comprising administering a single dose of a psychedelic compound which is psilocin or a prodrug of psilocin, or a pharmaceutically acceptable salt thereof, to said cancer patient, wherein:
said depression and/or anxiety is alleviated for at least one year; and
no further dose of the psychedelic compound is administered to said cancer patient for at least one year after administration of said single dose of the psychedelic compound.

2. The method of claim 1, wherein the psychedelic compound is psilocybin or a pharmaceutically acceptable salt thereof.

3. The method of claim 1, wherein the method further comprises providing the patient with psychotherapy treatment before, during, and/or after the administration of the single dose of the psychedelic compound.

4. The method of claim 3, wherein the psychotherapy treatment is provided in the three months before and/or the three months after administration of the single dose of the psychedelic compound.

5. The method of claim 1, wherein the psychotherapy treatment is selected from dyadic psychotherapy, supportive psychotherapy, attachment-based psychotherapy, behavioural therapy, body psychotherapy, somatic psychotherapy, brief therapy, cognitive analytical therapy, cognitive behaviour therapy, existential psychotherapy, gestalt therapy, humanistic integrative psychotherapy, hypno-psychotherapy, Jungian analysis, neuro-linguistic psychotherapy, object relations therapy, person-centered psychotherapy, psychodynamic psychotherapy or psychoanalysis, solution-focused brief therapy, transactional analysis, family systems therapy, internal family systems therapy, transpersonal psychotherapy, emotion-focused therapy, compassion-focused therapy, mindfulness-based cognitive therapy.

6. The method of claim 5, wherein the psychotherapy treatment is dyadic psychotherapy.

7. The method of claim 1, wherein the depression and/or anxiety is alleviated for at least three years.

8. The method of claim 7, wherein the depression and/or anxiety is alleviated for at least four and a half years.

9. The method of claim 1, wherein the cancer patient is suffering from stage I or stage II cancer.

10. The method of claim 1, wherein the cancer patient is suffering from late-stage cancer.

11. The method of claim 1, wherein the cancer patient is suffering from stage III or stage IV cancer.

12. The method of claim 1, wherein the cancer is selected from breast cancer, reproductive cancer, lymphoma, leukemia, acute lymphoblastic leukemia (ALL), acute myeloid leukemia (AML), adrenocortical carcinoma, anal cancer, appendix cancer, atypical teratoid/rhabdoid tumor, bile duct cancer, bladder cancer, bone cancer, brain cancer, Burkitt lymphoma, cervical cancer, chronic lymphocytic leukemia (CLL), chronic myelogenous leukemia (CML), chronic myeloproliferative neoplasms, colorectal cancer, cutaneous T-cell lymphoma, esophageal cancer, eye cancer, intraocular melanoma, fallopian tube cancer, gallbladder cancer, gastric (stomach) cancer, gastrointestinal carcinoid tumor, germ cell tumor, hairy cell leukemia, head and neck cancer, heart cancer, Hodgkin lymphoma, intraocular melanoma, islet cell tumor, kidney cancer, Langerhans cell histiocytosis, liver cancer, lung cancer (non-small cell, small cell, pleuropulmonary blastoma, and tracheobronchial tumor), melanoma, mesothelioma, metastatic cancer, mouth cancer, multiple endocrine neoplasia syndrome, multiple myeloma/plasma cell neoplasms, myelodysplastic syndrome, nasopharyngeal cancer, neuroblastoma, non-Hodgkin lymphoma, oral cancer, oropharyngeal cancer, osteosarcoma, ovarian cancer, pancreatic cancer, papillomatosis, paraganglioma, parathyroid cancer, penile cancer, pheochromocytoma, pituitary tumor, primary central nervous system (CNS) lymphoma, primary peritoneal cancer, prostate cancer, rectal cancer, retinoblastoma, salivary gland cancer, sarcoma, skin cancer, small intestine cancer, soft tissue sarcoma, testicular cancer, throat cancer, thymic carcinoma, thyroid cancer, urethral cancer, uterine cancer, and vaginal cancer.

13. The method of claim 12, wherein the cancer is selected from breast cancer, reproductive cancer, lymphoma, leukemia, bile duct cancer, brain cancer, esophageal cancer, gallbladder cancer, head and neck cancer, heart cancer, liver cancer, lung cancer, mesothelioma and pancreatic cancer.

14. The method of claim 13, wherein the cancer is selected from breast cancer, leukemia, lymphoma, and reproductive cancer.

15. The method of claim 1, wherein the patient has an anxiety-related diagnosis.

16. The method of claim 15, wherein the anxiety-related diagnosis is selected from an adjustment disorder, panic disorder, post-traumatic stress disorder, obsessive-compulsive disorder, social phobia, acute stress disorder, and generalized anxiety disorder.

17. The method of claim 16, wherein the anxiety-related diagnosis is an adjustment disorder or generalized anxiety disorder.

18. The method of claim 1, wherein a total Hospital Anxiety and Depression Scale score of said cancer patient is maintained below about 12 for at least one year.

19. The method of claim 1, wherein a Beck Depression Inventory-II score of said cancer patient is maintained below about 12 for at least one year.

20. The method of claim 1, wherein the single dose of the psychedelic compound is from about 10 mg to about 40 mg.

21. The method of claim 20, wherein the single dose of the psychedelic compound is from about 20 mg to about 30 mg.

22. The method of claim 21, wherein the single dose of the psychedelic compound is about 25 mg.

23. A method of reducing death anxiety in a cancer patient, the method comprising administering a single dose of a psychedelic compound which is psilocin or a prodrug of psilocin, or a pharmaceutically acceptable salt thereof, to said cancer patient, wherein:
said death anxiety is reduced for at least one year; and
no further dose of the psychedelic compound is administered to said cancer patient for at least one year after administration of said single dose of the psychedelic compound.

24. A method of reducing demoralization in a cancer patient, the method comprising administering a single dose of a psychedelic compound which is psilocin or a prodrug of psilocin, or a pharmaceutically acceptable salt thereof, to said cancer patient, wherein:
said demoralization is reduced for at least one year; and
no further dose of the psychedelic compound is administered to said cancer patient for at least one year after administration of said single dose of the psychedelic compound.

25. The method of claim 24, wherein a Demoralization Scale score of said cancer patient is maintained below about 30 for at least one year.

26. A method of reducing hopelessness in a cancer patient, the method comprising administering a single dose of a psychedelic compound which is psilocin or a prodrug of psilocin, or a pharmaceutically acceptable salt thereof, to said cancer patient, wherein:
said hopelessness is reduced for at least one year; and
no further dose of the psychedelic compound is administered to said cancer patient for at least one year after administration of said single dose of the psychedelic compound.

27. The method of claim 26, wherein a Hopelessness Assessment in Illness score of said cancer patient is maintained below about 8 for at least one year.

28. A kit comprising:
a single dose of a psychedelic compound which is psilocin or a prodrug of psilocin, or a pharmaceutically acceptable salt thereof; and
instructions for use of said single dose of the psychedelic compound in a method of alleviating depression and/or anxiety in a cancer patient, the method comprising administering the single dose of the psychedelic compound to said cancer patient, wherein: said depression and/or anxiety is alleviated for at least one year; and no further dose of the psychedelic compound is administered to said cancer patient for at least one year after administration of said single dose of the psychedelic compound.
